(19) United States
(12) Patent Application Publication
(10) Pub. No.: US 2023/0172483 A1
(43) Pub. Date: Jun. 8, 2023

(54) WEARABLE ELECTRONIC DEVICE AND METHOD OF OPERATING THE SAME

(71) Applicant: SAMSUNG ELECTRONICS CO., LTD., Suwon-si (KR)

(72) Inventors: Hyunjun JUNG, Suwon-si (KR); Sunghwan CHO, Suwon-si (KR)

(21) Appl. No.: 18/102,937

(22) Filed: Jan. 30, 2023

Related U.S. Application Data
(63) Continuation of application No. PCT/KR2022/012496, filed on Aug. 22, 2022.

(30) Foreign Application Priority Data
Oct. 25, 2021 (KR) 10-2021-0142846
Nov. 17, 2021 (KR) 10-2021-0158423

(51) Int. Cl. A61B 5/08; A61B 5/00; A61B 5/11; A61B 5/024

(52) U.S. Cl. CPC A61B 5/0816; A61B 5/681; A61B 5/1116; A61B 5/02405; A61B 2562/0219

(57) ABSTRACT

A wearable electronic device according to an embodiment includes: a sensor module including a first sensor configured to sense a first signal including a pulse wave based on a respiration of a user corresponding to a first time, the respiration including an inhalation and an exhalation, and a second sensor configured to sense a second signal including a first pattern corresponding to the inhalation and a second pattern corresponding to the exhalation. The wearable electronic device includes a processor configured to: match a first respiratory characteristic of the first signal and a second respiratory characteristic of the second signal based on a correlation between the first signal and the second signal, and estimate a respiration phase of the user corresponding to the second signal measured at a second time after the first time, based on the matched first and second respiratory characteristics.

### CROSS-REFERENCE TO RELATED APPLICATIONS

This application is a continuation of International Application No. PCT/KR2022/012496 designating the United States, filed on Aug. 22, 2022, in the Korean Intellectual Property Receiving Office and claiming priority to Korean Patent Application No. 10-2021-0142846, filed on Oct. 25, 2021, in the Korean Intellectual Property Office, and to Korean Patent Application No. 10-2021-0158423, filed on Nov. 17, 2021, in the Korean Intellectual Property Office, the disclosures of all of which are incorporated by reference herein in their entireties.

### BACKGROUND

### 1. Field

The disclosure relates to a wearable electronic device and a method of operating the wearable electronic device.

### 2. Description of Related Art

A wearable electronic device may refer to an electronic device that is used in close contact with a user's body beyond a portable device, for example, a smartphone or a notebook computer. The wearable electronic device may take the form of, for example, glasses, a watch, or a head-mounted display (HMD), and may be connected to a smartphone or may independently perform various functions. Unlike smartphones or notebook computers, which need to be taken out and checked all the time, users can more conveniently perform various functions, for example, simple checking of text messages or e-mails, health management such as checking of a heart rate and calculating of an exercise amount, an exercise function, and schedule management, using wearable electronic devices.

In a phenomenon in which biosignals are modulated by respiration (e.g., a phenomenon in which an interval of a photoplethysmogram (PPG) signal is reduced and an amplitude and a baseline decrease during inhalation), respiration information about whether a respiration of a user is inhalation or exhalation may be analyzed based on a change in a shape of a PPG signal. In general, a heart rate of 50 beats per minute (BPM) may be maintained. If the heart rate is converted into an interval between beats, a period slightly less than one second may be set. If a respiration of a user is fast, a possibility of incorrectly measuring respiration information may increase due to an extremely low bit rate for sampling. If a physiological change due to a respiration is not measured directly from a body part of a user wearing a wearable electronic device, only a movement caused by the respiration may be measured, and accordingly it may be difficult to distinguish between inhalation and exhalation.

### SUMMARY

Embodiments of the disclosure provide a device and method in which respiration phases of a user including inhalation and exhalation may be distinguished with a high accuracy.

Embodiments of the disclosure provide a device and method in which a respiration phase of a user may be detected in real time by an accelerometer (ACC) signal having a high signal-to-noise ratio (SNR) and a fast reaction rate.

Embodiments of the disclosure provide a device and method in which a respiration phase of a user may be quickly and accurately detected by respiratory characteristics previously analyzed through matching of an ACC signal and a PPG signal.

According to an example embodiment, a wearable electronic device may include: a sensor module including a first sensor configured to sense a first signal including a pulse wave based on a respiration corresponding to a first time, the respiration including inhalation and exhalation, and a second sensor configured to sense a second signal including a first pattern corresponding to the inhalation and a second pattern corresponding to the exhalation, and a processor configured to: match a first respiratory characteristic of the first signal and a second respiratory characteristic of the second signal based on a correlation between the first signal and the second signal, and estimate a respiration phase corresponding to the second signal measured at a second time after the first time based on the matched first and second respiratory characteristics.

According to an example embodiment, a method of operating a wearable electronic device may include: collecting, from a sensor module a first signal including a change in a heart rate based on a respiration sensed at a first time, and a second signal including a first pattern corresponding to inhalation and a second pattern corresponding to exhalation, the respiration including the inhalation and the exhalation, matching a first respiratory characteristic of the first signal and a second respiratory characteristic of the second signal based on a correlation between the first signal and the second signal, and estimating a respiration phase corresponding to the second signal measured at a second time after the first time based on the matched first and second respiratory characteristics.

According to an example embodiment, a wearable electronic device may more quickly and accurately detect a respiration phase of a user based on respiratory characteristics analyzed in advance through matching of an ACC signal and a PPG signal.

According to an example embodiment, a wearable electronic device may provide a guide to allow a user to accurately perform a breathing exercise and may feed back a result of the breathing exercise to help the user manage mental health such as stress reduction.

According to an example embodiment, a wearable electronic device may more rapidly provide medical information for medical diagnosis by estimating a respiration phase of a user in real time.

### BRIEF DESCRIPTION OF THE DRAWINGS

The above and other aspects, features, and advantages of certain embodiments of the present disclosure will be more apparent from the following detailed description, taken in conjunction with the accompanying drawings, in which:

FIG. 1 is a block diagram illustrating an example electronic device in a network environment according to an embodiment;

FIGS. 2A and 2B are front and rear perspective views, respectively, of an electronic device according to an embodiment;

FIG. 3 is an exploded perspective view of an electronic device according to an embodiment;

FIG. 4 is a block diagram illustrating an example configuration of a wearable electronic device according to an embodiment;

FIG. 5 is a diagram illustrating an example operation of a wearable electronic device according to an embodiment;

FIG. 6 is a flowchart illustrating an example method of operating a wearable electronic device according to an embodiment;

FIG. 7 is a flowchart illustrating an example method of operating a wearable electronic device according to an embodiment;

FIG. 8 illustrates graphs of signals measured at respiratory rates at different intervals in a wearable electronic device according to an embodiment; and

FIG. 9 is a diagram illustrating an example method of performing a breathing exercise using a wearable electronic device according to an embodiment.

### DETAILED DESCRIPTION

Hereinafter, embodiments will be described in greater detail with reference to the accompanying drawings. When describing the embodiments with reference to the accompanying drawings, like reference numerals refer to like elements and a repeated description related thereto may not be provided.

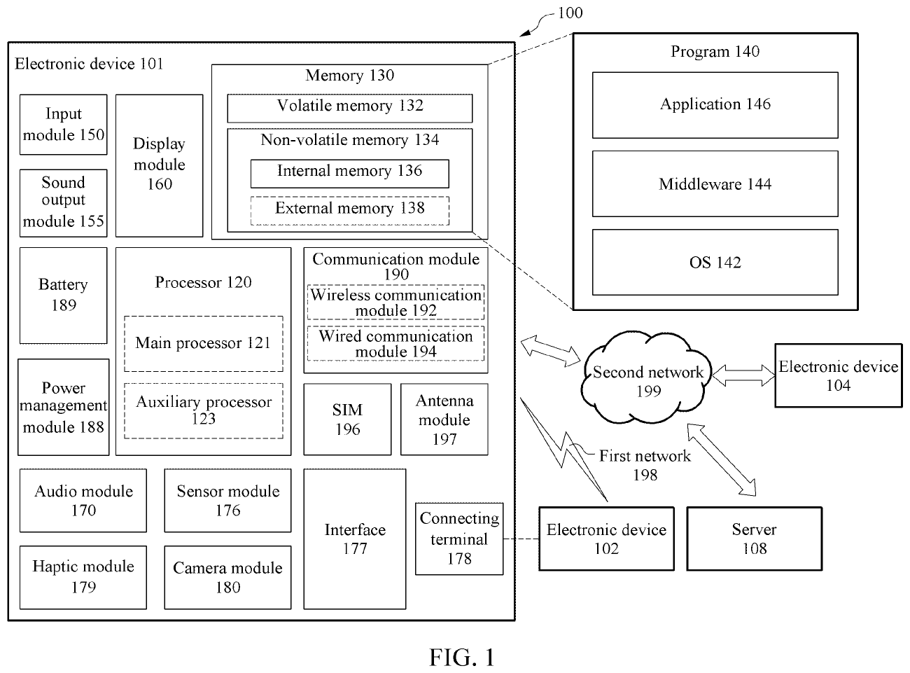

FIG. 1 is a block diagram illustrating an example electronic device 101 in a network environment 100 according to an embodiment. Referring to FIG. 1, the electronic device 101 in the network environment 100 may communicate with an electronic device 102 via a first network 198 (e.g., a short-range wireless communication network), or communicate with at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, a memory 130, an input module 150, a sound output module 155, a display module 160, an audio module 170, and a sensor module 176, an interface 177, a connecting terminal 178, a haptic module 179, a camera module 180, a power management module 188, a battery 189, a communication module 190, a subscriber identification module (SIM) 196, or an antenna module 197. In various embodiments, at least one of the components (e.g., the connecting terminal 178) may be omitted from the electronic device 101, or one or more other components may be added in the electronic device 101. In various embodiments, some of the components (e.g., the sensor module 176, the camera module 180, or the antenna module 197) may be integrated as a single component (e.g., the display module 160).

The processor 120 may execute, for example, software (e.g., a program 140) to control at least one other component (e.g., a hardware or software component) of the electronic device 101 connected to the processor 120, and may perform various data processing or computation. According to an embodiment, as at least a part of data processing or computation, the processor 120 may store a command or data received from another component (e.g., the sensor module 176 or the communication module 190) in a volatile memory 132, process the command or the data stored in the volatile memory 132, and store resulting data in a non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with the main processor 121. For example, when the electronic device 101 includes the main processor 121 and the auxiliary processor 123, the auxiliary processor 123 may be adapted to consume less power than the main processor 121 or to be specific to a specified function. The auxiliary processor 123 may be implemented separately from the main processor 121 or as a part of the main processor 121.

The auxiliary processor 123 may control at least some of functions or states related to at least one (e.g., the display module 160, the sensor module 176, or the communication module 190) of the components of the electronic device 101, instead of the main processor 121 while the main processor 121 is in an inactive (e.g., sleep) state or along with the main processor 121 while the main processor 121 is an active state (e.g., executing an application). According to an embodiment, the auxiliary processor 123 (e.g., an ISP or a CP) may be implemented as a portion of another component (e.g., the camera module 180 or the communication module 190) that is functionally related to the auxiliary processor 123. According to an embodiment, the auxiliary processor 123 (e.g., an NPU) may include a hardware structure specified for artificial intelligence model processing. An artificial intelligence model may be generated by machine learning. Such learning may be performed by, for example, the electronic device 101 in which artificial intelligence is performed, or performed via a separate server (e.g., the server 108). Learning algorithms may include, but are not limited to, for example, supervised learning, unsupervised learning, semi-supervised learning, or reinforcement learning. The AI model may include a plurality of artificial neural network layers. An artificial neural network may include, for example, a deep neural network (DNN), a convolutional neural network (CNN), a recurrent neural network (RNN), a restricted Boltzmann machine (RBM), a deep belief network (DBN), and a bidirectional recurrent deep neural network (BRDNN), a deep Q-network, or a combination of two or more thereof, but is not limited thereto. The AI model may additionally or alternatively include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored as software in the memory 130, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output a sound signal to the outside of the electronic device 101. The sound output module 155 may include, for example, a speaker or a receiver. The speaker may be used for general purposes, such as playing multimedia or playing record. The receiver may be used to receive an incoming call. According to an embodiment, the receiver may be implemented separately from the speaker or as a part of the speaker.

The display module 160 may visually provide information to the outside (e.g., a user) of the electronic device 101. The display module 160 may include, for example, a control circuit for controlling a display, a hologram device, or a projector and control circuitry to control a corresponding one of the display, the hologram device, and the projector. According to an embodiment, the display module 160 may include a touch sensor adapted to detect a touch, or a pressure sensor adapted to measure the intensity of force incurred by the touch.

The audio module 170 may convert a sound into an electric signal or vice versa. According to an embodiment, the audio module 170 may obtain the sound via the input module 150 or output the sound via the sound output module 155 or an external electronic device (e.g., the electronic device 102 such as a speaker or a headphone) directly or wirelessly connected to the electronic device 101.

The sensor module 176 may detect an operational state (e.g., power or temperature) of the electronic device 101 or an environmental state (e.g., a state of a user) external to the electronic device 101, and generate an electric signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., by wire) or wirelessly. According to an embodiment, the interface 177 may include, for example, a high-definition multimedia interface (HDMI), a universal serial bus (USB) interface, a secure digital (SD) card interface, or an audio interface.

The connecting terminal 178 may include a connector via which the electronic device 101 may be physically connected to an external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electric signal into a mechanical stimulus (e.g., a vibration or a movement) or an electrical stimulus which may be recognized by a user via his or her tactile sensation or kinesthetic sensation. According to an embodiment, the haptic module 179 may include, for example, a motor, a piezoelectric element, or an electric stimulator.

The camera module 180 may capture a still image and moving images. According to an embodiment, the camera module 180 may include one or more lenses, image sensors, image signal processors, or flashes.

The power management module 188 may manage power supplied to the electronic device 101. According to an embodiment, the power management module 188 may be implemented as, for example, at least a part of a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more CPs that are operable independently of the processor 120 (e.g., an AP) and that support a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module, or a power line communication (PLC) module). A corresponding one of these communication modules may communicate with the external electronic device 104 via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., a LAN or a wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the SIM 196.

The wireless communication module 192 may support a 5G network after a 4G network, and a next-generation communication technology, e.g., a new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., a mmWave band) to achieve, e.g., a high data transmission rate. The wireless communication module 192 may support various technologies for securing performance on a high-frequency band, such as, e.g., beamforming, massive multiple-input and multiple-output (MIMO), full dimensional MIMO (FD-MIMO), an array antenna, analog beam-forming, or a large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in a communication network, such as the first network 198 or the second network 199, may be selected by, for example, the communication module 190 from the plurality of antennas. The signal or the power may be transmitted or received between the communication module 190 and the external electronic device via the at least one selected antenna. According to an embodiment, another component (e.g., a radio frequency integrated circuit (RFIC)) other than the radiating element may be additionally formed as a part of the antenna module 197.

According to an embodiment, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the external electronic devices 102 or 104 may be a device of the same type as or a different type from the electronic device 101. According to an embodiment, all or some of operations to be executed by the electronic device 101 may be executed at one or more external electronic devices (e.g., the external devices 102 and 104, and the server 108). For example, if the electronic device 101 needs to perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and may transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

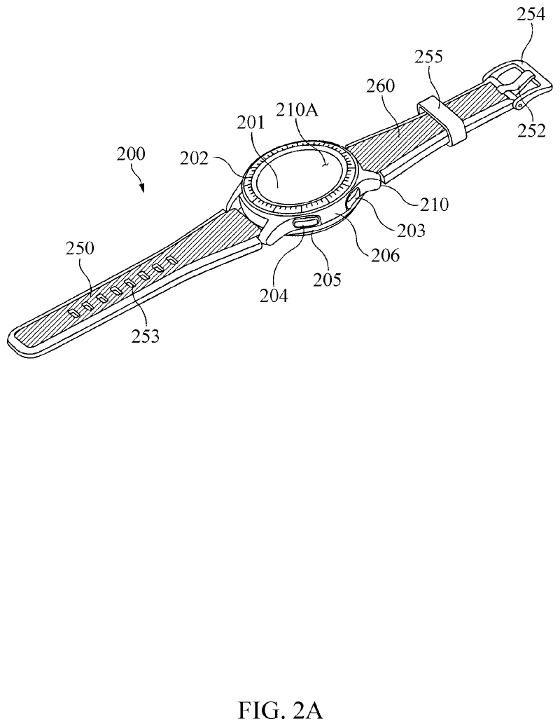

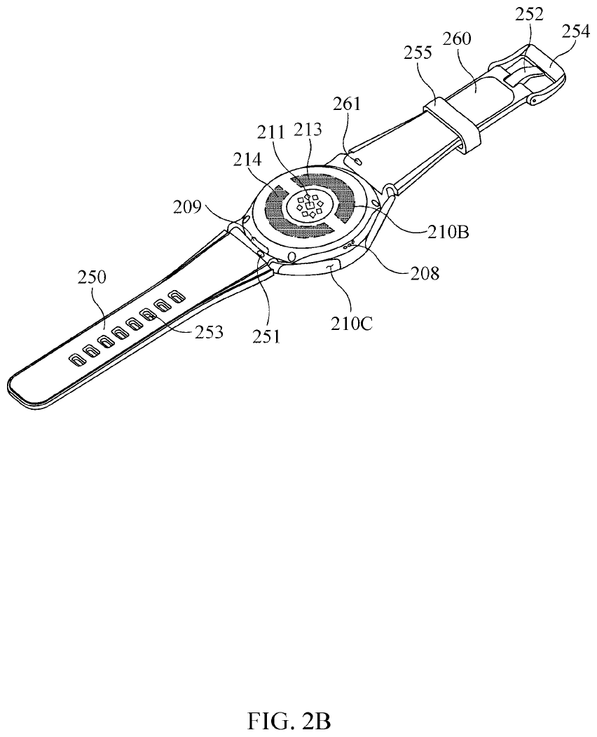

FIGS. 2A and 2B are front and rear perspective views, respectively, of an electronic device according to various embodiments. Referring to FIGS. 2A and 2B, according to an embodiment, an electronic device 200 (e.g., the electronic device 101 of FIG. 1) may include a housing 210 including a first surface (or a front surface) 210A, a second surface (or a rear surface) 210B, and a side surface 210C surrounding a space between the first surface 210A and the second surface 210B, and fastening members 250 and 260 connected to at least a portion of the housing 210 and configured to detachably attach the electronic device 200 to a body part (e.g., a wrist, or an ankle) of a user. In an embodiment (not shown), the housing may also refer to a structure which forms a portion of the first surface 210A, the second surface 210B, and the side surface 210C of FIG. 2A. According to an embodiment, the first surface 210A may be formed by a front plate 201 (e.g., a glass plate or a polymer plate including various coating layers) of which at least a portion is substantially transparent. The second surface 210B may be formed by a rear plate 207 that is substantially opaque. The rear plate 207 may be formed of, for example, coated or colored glass, ceramic, polymer, metal (e.g., aluminum, stainless steel (SS), or magnesium), or a combination of at least two thereof. The side surface 210C may be coupled to the front plate 201 and the rear plate 207 and may be formed by a side bezel structure (or a “side member”) 206 including a metal and/or a polymer. In an embodiment, the rear plate 207 and the side bezel structure 206 may be integrally formed and may include the same material (e.g., a metal material such as aluminum). The fastening members 250 and 260 may be formed of various materials and may have various shapes. For example, the fastening members 250 and 260 may be formed of woven fabric, leather, rubber, urethane, metal, ceramic, or a combination of at least two of the aforementioned materials and may be implemented in an integrated form or with a plurality of unit links that are movable relative to each other.

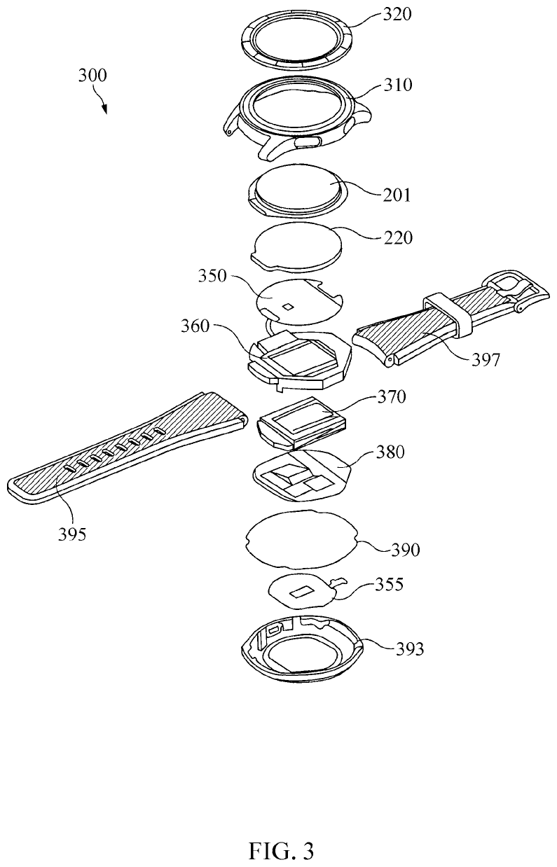

According to an embodiment, the electronic device 200 may include at least one of a display 220 (refer to FIG. 3), audio modules 205 and 208, a sensor module 211, key input devices 202, 203, and 204, and a connector hole 209. In an embodiment, the electronic device 200 may not include at least one (e.g., the key input devices 202, 203, and 204, the connector hole 209, or the sensor module 211) of the components, or additionally include other components.

The display 220 (refer to FIG. 3) may be visible through, for example, some portions of the front plate 201. The display 220 may have a shape corresponding to a shape of the front plate 201, and may have various shapes such as a circle, an oval, or a polygon. The display 220 may be coupled to or disposed adjacent to a touch sensing circuit, a pressure sensor capable of measuring an intensity (or pressure) of a touch, and/or a fingerprint sensor.

The audio modules 205 and 208 may include a microphone hole 205 and a speaker hole 208. A microphone for acquiring an external sound may be disposed in the microphone hole 205. In some embodiments, a plurality of microphones may be disposed to detect a direction of a sound. The speaker hole 208 may be used as an external speaker and a call receiver for calls. In an embodiment, the speaker hole 208 and the microphone hole 205 may be implemented as a single hole, or a speaker (e.g., a piezo speaker) may be included without the speaker hole 208

The sensor module 211 may generate an electrical signal or a data value corresponding to an internal operating state of the electronic device 200 or an external environmental state. The sensor module 211 may include, for example, a biometric sensor module 211 (e.g., a heart rate monitor (HRM) sensor) disposed on the second surface 210B of the housing 210. The electronic device 200 may further include at least one of sensor modules (not shown), for example, a gesture sensor, a gyro sensor, an atmospheric pressure sensor, a magnetic sensor, an acceleration sensor, a grip sensor, a color sensor, an infrared (IR) sensor, a biometric sensor, a temperature sensor, a humidity sensor, or an illuminance sensor.

The sensor module 211 may include electrode areas 213 and 214 that form a portion of the surface of the electronic device 200 and a biosignal detection circuit (not shown) electrically connected to the electrode areas 213 and 214. For example, the electrode areas 213 and 214 may include a first electrode area 213 and a second electrode area 214 disposed on the second surface 210B of the housing 210. The sensor module 211 may be configured such that the electrode areas 213 and 214 obtain an electrical signal from a body part of the user, and the biosignal detection circuit detects biometric information of the user based on the electrical signal.

The key input devices 202, 203, and 204 may include a wheel key 202 disposed on the first surface 210A of the housing 210 and rotatable in at least one direction, and/or side key buttons 203 and 204 disposed on the side surface 210C of the housing 210. The wheel key 202 may have a shape corresponding to the shape of the front plate 201. In an embodiment, the electronic device 200 may not include some or all of the above-described key input devices 202, 203, and 204, and the key input devices 202, 203, and 204 that are not included may be implemented in other forms such as soft keys on the display 220. The connector hole 209 may include another connector hole (not shown) that accommodates a connector (e.g., a universal serial bus (USB) connector) for transmitting and receiving power and/or data to and from an external electronic device and accommodates a connector for transmitting and receiving an audio signal to and from an external electronic device. The electronic device 200 may further include, for example, a connector cover (not shown) that covers at least a portion of the connector hole 209 and blocks infiltration of external foreign materials into the connector hole 209.

The fastening members 250 and 260 may be detachably fastened to at least a partial area of the housing 210 using locking members 251 and 261. The fastening members 250 and 260 may include one or more of a fixing member 252, a fixing member fastening hole 253, a band guide member 254, and a band fixing ring 255.

The fixing member 252 may be configured to fix the housing 210 and the fastening members 250 and 260 to a part (e.g., a wrist, an ankle, etc.) of the user's body. The fixing member fastening hole 253 may correspond to the fixing member 252 to fix the housing 210 and the fastening members 250 and 260 to the part of the user's body. The band guide member 254 may be configured to limit a range of a movement of the fixing member 252 when the fixing member 252 is fastened to the fixing member fastening hole 253, so that the fastening members 250 and 260 may be fastened to the part of the user's body in a state of being brought into close contact with the part of the user's body. The band fixing ring 255 may limit a range of a movement of the fastening member 250, 260 in a state in which the fixing member 252 and the fixing member fastening hole 253 are fastened with each other.

FIG. 3 is an exploded perspective view of an electronic device according to various embodiments. Referring to FIG. 3, an electronic device 300 (e.g., the electronic device 101 of FIG. 1 or the electronic device 200 of FIGS. 2A and 2B) may include a side bezel structure 310, a wheel key 320, a front plate 201, a display 220, a first antenna 350, a second antenna 355, a support member 360 (e.g., a bracket), a battery 370, a PCB 380, a sealing member 390, a rear plate 393, and fastening members 395 and 397. At least one of the components of the electronic device 300 may be the same as or similar to at least one of the components of the electronic device 100 of FIG. 1, or the electronic device 200 of FIGS. 2A and 2B, and a repeated description thereof will be omitted hereinafter.

The support member 360 may be disposed inside the electronic device 300 and connected to the side bezel structure 310, or may be integrally formed with the side bezel structure 310. The support member 360 may be formed of, for example, a metal material and/or a non-metal material (e.g., polymer). The display 220 may be connected to one surface of the support member 360, and the PCB 380 may be connected to another surface of the support member 360.

The PCB 380 may be provided with a processor, a memory, and/or an interface mounted thereon. The processor may include, for example, one or more of a CPU, an AP, a GPU, a sensor processor, or a CP. The memory may include, for example, a volatile memory or a non-volatile memory. The interface may include, for example, an HDMI, a USB interface, an SD card interface, or an audio interface. For example, the interface may electrically or physically connect the electronic device 300 to an external electronic device, and may include a USB connector, an SD card/multimedia card (MMC) connector, or an audio connector.

The battery 370, which is a device for supplying power to at least one component of the electronic device 300, may include, for example, a non-rechargeable primary battery, a rechargeable secondary battery, or a fuel cell. For example, at least a portion of the battery 370 may be disposed on substantially the same plane as the PCB 380. The battery 370 may be disposed integrally inside the electronic device 300, or disposed detachably from the electronic device 300.

The first antenna 350 may be disposed between the display 220 and the support member 360. The first antenna 350 may include, for example, a near-field communication (NFC) antenna, a wireless charging antenna, and/or a magnetic secure transmission (MST) antenna. For example, the first antenna 350 may perform short-range communication with an external device, wirelessly transmit and receive power used for charging, or transmit a magnetism-based signal including a short-range communication signal or payment data. In an embodiment, an antenna structure may be formed by a portion of the side bezel structure 310 and/or the support member 360, or a combination thereof.

The second antenna 355 may be disposed between the PCB 380 and the rear plate 393. The second antenna 355 may include, for example, an NFC antenna, a wireless charging antenna, and/or an MST antenna. For example, the second antenna 355 may perform short-range communication with an external device, wirelessly transmit and receive power used for charging, or transmit a magnetism-based signal including a short-range communication signal or payment data. In an embodiment, an antenna structure may be formed by a portion of the side bezel structure 310 and/or the rear plate 393, or a combination thereof.

The sealing member 390 may be disposed between the side bezel structure 310 and the rear plate 393. The sealing member 390 may be configured to prevent and/or reduce moisture and foreign materials from being introduced into a space surrounded by the side bezel structure 310 and the rear plate 393 from the outside.

The electronic devices according to an embodiment may be various types of electronic devices. The electronic device may include, for example, a portable communication device (e.g., a smartphone), a computer device, a portable multimedia device, a portable medical device, a camera, a wearable device, a home appliance device, or the like. According to an embodiment of the disclosure, the electronic device is not limited to those described above.

It should be appreciated that embodiments of the present disclosure and the terms used therein are not intended to limit the technological features set forth herein to particular embodiments and include various changes, equivalents, or replacements for a corresponding embodiment. In connection with the description of the drawings, like reference numerals may be used for similar or related components. It is to be understood that a singular form of a noun corresponding to an item may include one or more of the things, unless the relevant context clearly indicates otherwise. As used herein, “A or B”, “at least one of A and B”, “at least one of A or B”, “A, B or C”, “at least one of A, B and C”, and “at least one of A, B, or C,” each of which may include any one of the items listed together in the corresponding one of the phrases, or all possible combinations thereof. Terms such as “first”, “second”, or “first” or “second” may simply be used to distinguish the component from other components in question, and may refer to components in other aspects (e.g., importance or order) is not limited. It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., by wire), wirelessly, or via a third element.

As used in connection with an embodiment of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

An embodiment as set forth herein may be implemented as software (e.g., the program 140) including one or more instructions that are stored in a storage medium (e.g., an internal memory 136 or an external memory 138) that is readable by a machine (e.g., the electronic device 101 of FIG. 1). For example, a processor (e.g., the processor 120) of the machine (e.g., the electronic device 101) may invoke at least one of the one or more instructions stored in the storage medium, and execute it. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Here, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to an embodiment of the disclosure may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities, and some of the multiple entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to an embodiment, the integrated component may still perform one or more functions of each of the plurality of components in the same or similar manner as they are performed by a corresponding one of the plurality of components before the integration. According to an embodiment, operations performed by the module, the program, or another component may be carried out sequentially, in parallel, repeatedly, or heuristically, or one or more of the operations may be executed in a different order or omitted, or one or more other operations may be added.

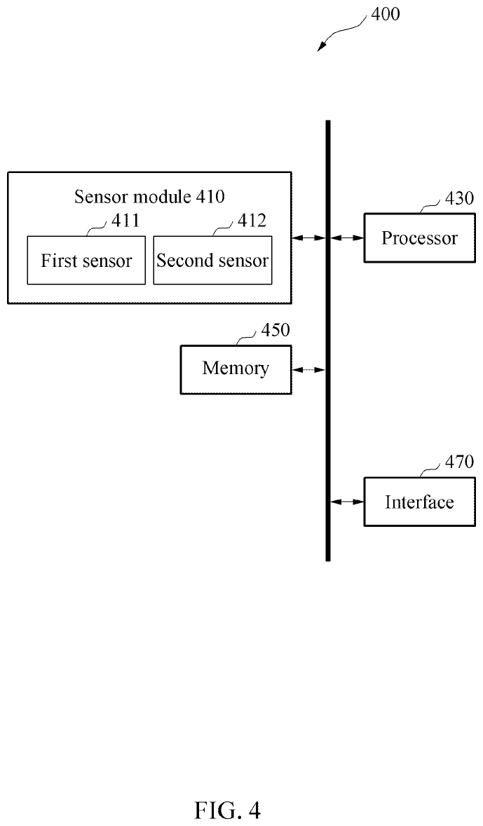

FIG. 4 is a block diagram illustrating an example configuration of a wearable electronic device according to an embodiment. Referring to FIG. 4, a wearable electronic device 400 (e.g., the electronic device 101 of FIG. 1, the electronic device 200 of FIGS. 2A and 2B, or the electronic device 300 of FIG. 3) according to an embodiment may include a sensor module (e.g., including at least one sensor) 410 (e.g., the sensor module 176 of FIG. 1, and the sensor module 211 of FIGS. 2A and 2B), and a processor (e.g., including processing circuitry) 430 (e.g., the processor 120 of FIG. 1). The wearable electronic device 400 may further include a memory 450 (e.g., the memory 130 of FIG. 1), and an interface (e.g., including interface circuitry) 470 (e.g., the interface 177 of FIG. 1).

For example, the sensor module 410 may include a first sensor 411 and a second sensor 412, however, the embodiments are not limited thereto.

The first sensor 411 may sense a first signal including a pulse wave according to a respiration of a user corresponding to a first time. The respiration of the user may include inhalation and exhalation. The inhalation may be performed by inhaling of a user, and may also be referred to as “inspiration”, which is a breath in which air is inhaled. The exhalation may be performed by exhaling of a user, and may also be referred to as “expiration”, which is a breath in which air is exhaled.

The first sensor 411 may be, for example, a photoplethysmogram (PPG) sensor in which an LED reflects light from one side and travels to another side and a detector receives the light bounced from the other side to estimate a blood flow rate, but is not limited thereto. For example, the PPG sensor may determine that the blood flow rate is high, that is, a pulse wave is great, in response to a small amount of light to be detected, and that the blood flow rate is low, that is, a pulse wave is small, in response to a large amount of light to be detected.

The second sensor 412 may sense a second signal including a first pattern corresponding to the inhalation and a second pattern corresponding to the exhalation according to the respiration of the user. The second sensor 412 may include, for example, at least one of an acceleration sensor configured to sense a change in an acceleration according to a respiration and a movement of a user, a gyro sensor configured to sense a change in a rotating angular speed according to the respiration and movement of the user, an acoustic sensor configured to sense sounds corresponding to inhalation and exhalation, or a radio frequency (RF) sensor configured to sense a change in a shape of a chest of the user that is changed by the respiration of the user, by an RF signal, but is not necessarily limited thereto.

According to an embodiment, the wearable electronic device 400 may further enhance an accuracy of the second signal sensed by the second sensor 412 through another wearable device located in a position in which a movement of a chest of the user may be sensed according to the respiration. For example, the wearable electronic device 400 may sense the movement of the chest using an accelerometer of a virtual reality (VR) device or an accelerometer mounted in earbuds under an assumption that there is no head motion accompanied by the respiration, and may connect the movement of the chest to PPG-based inhalation/exhalation signals measured substantially simultaneously by the wearable electronic device 400, to enhance an accuracy of measurement by an accelerometer (ACC) signal having a higher signal-to-noise ratio (SNR).

The second signal may include various signals that may be classified into patterns respectively corresponding to inhalation and exhalation according to a respiration of a user, as described above. For example, a pattern of a sound generated when a user inhales and a pattern of a sound generated when the user exhales may be distinguished from each other. In addition, a rising pattern of an acceleration signal corresponding to a movement of a lung of a user and/or a movement of a body of the user generated during the inhalation, and a falling pattern of an acceleration signal corresponding to a movement of the lung of the user and/or a movement of the body of the user generated during the exhalation may be distinguished from each other. The second signal may include all such various signals with distinguishable patterns generated for each of the inhalation and the exhalation.

In an embodiment, an example in which the first sensor 411 and/or the second sensor 412 are included in the wearable electronic device 400 is described for convenience of description, however, the embodiments are not limited thereto. The wearable electronic device 400 may also be used to estimate a respiration phase by remotely measuring a first signal (e.g., a PPG signal) and remotely measuring a motion signal of a subject in the same manner of measuring a pulse wave using a camera.

The processor 430 may include various processing circuitry and match a first respiratory characteristic of the first signal and a second respiratory characteristic of the second signal based on a correlation between the first signal and the second signal. The processor 430 may extract the first respiratory characteristic from the first signal. The processor 430 may sample the first signal, and may extract the first respiratory characteristic including an inhalation interval and an exhalation interval of the first signal based on at least one of an interval in which a heart rate variability (HRV) by the sampled first signal increases, or an interval in which a stroke volume variation by the sampled first signal decreases.

The processor 430 may extract the second respiratory characteristic from the second signal. The processor 430 may determine a pattern corresponding to a rising interval and a pattern corresponding to a falling interval in the second signal among the first pattern and the second pattern, based on a slope of each of the first pattern and the second pattern of the second signal.

In an embodiment, a respiratory characteristic may correspond to, for example, a rising interval or a falling interval in a signal waveform, may correspond to an inhalation interval or an exhalation interval, and may also correspond to an inhalation sound interval or an exhalation sound interval. Hereinafter, the first respiratory characteristic may refer to a respiration-related characteristic extracted from the first signal. The second respiratory characteristic may refer to a respiration-related characteristic extracted from the second signal.

The processor 430 may match the first respiratory characteristic and the second respiratory characteristic based on a comparison result between the first respiratory characteristic and the second respiratory characteristic. In an example, the processor 430 may match the first respiratory characteristic and the second respiratory characteristic based on a correlation, for example, which of the first pattern and the second pattern of the second signal a portion of the first signal determined as an inhalation interval overlaps more. If the portion of the first signal determined as the inhalation interval overlaps the second pattern of the second signal more, the processor 430 may match the second pattern of the second signal to the inhalation interval and match the first pattern of the second signal to the exhalation interval. In another example, the processor 430 may match the first respiratory characteristic and the second respiratory characteristic based on a correlation, for example, which of the first pattern and the second pattern of the second signal a portion of the first signal determined as an exhalation interval overlaps more. For example, when the portion of the first signal determined as the exhalation interval more overlaps the second pattern of the second signal, the processor 430 may match the second pattern of the second signal to the exhalation interval and match the first pattern of the second signal to the inhalation interval.

For example, the first signal may be a PPG signal, and the second signal may be an ACC signal. In this example, the processor 430 may match an inhalation interval of the PPG signal to a rising interval of the ACC signal and match an exhalation interval of the PPG signal to a falling interval of the ACC signal, based on a correlation between the PPG signal and the ACC signal.

Matching the first respiratory characteristic and the second respiratory characteristic by the processor 430 may correspond to a “learning process” of identifying respiratory characteristics of a user in advance. The processor 430 may more quickly and accurately estimate a respiration phase of the user based on the second signal sensed at the second time by respiratory characteristics of the user identified in advance by signals sensed at the first time.

The processor 430 may estimate a respiration phase of the user corresponding to the second signal measured at the second time after the first time, based on the matched first and second respiratory characteristics. The processor 430 may convert a rising interval of the second signal measured at the second time to an inhalation interval and convert a falling interval of the second signal measured at the second time to an exhalation interval, based on the matched first and second respiratory characteristics. The processor 430 may estimate the respiration phase of the user based on the inhalation interval and the exhalation interval. For example, the processor 430 may convert a rising interval of an ACC signal measured at the second time to an inhalation interval and convert a falling interval of the ACC signal to an exhalation interval, based on a result obtained by matching respiratory characteristics between the ACC signal and the PPG signal sensed at the first time, to estimate the respiration phase corresponding to the second time. The respiration phase may be divided into an inhalation interval and an exhalation interval.

In addition, the processor 430 may determine whether a posture of the user is changed based on the second signal measured at the second time, and may estimate the respiration phase of the user corresponding to the second time based on whether the posture of the user is changed. The processor 430 may determine whether the posture of the user is changed based on a comparison result between a third signal generated by an arbitrary combination of detailed signals included in the second signal and a threshold. Here, the threshold may correspond to a signal of an interval (threshold interval) having a low slope in which a user temporarily stops breathing during a transition between exhalation and inhalation.

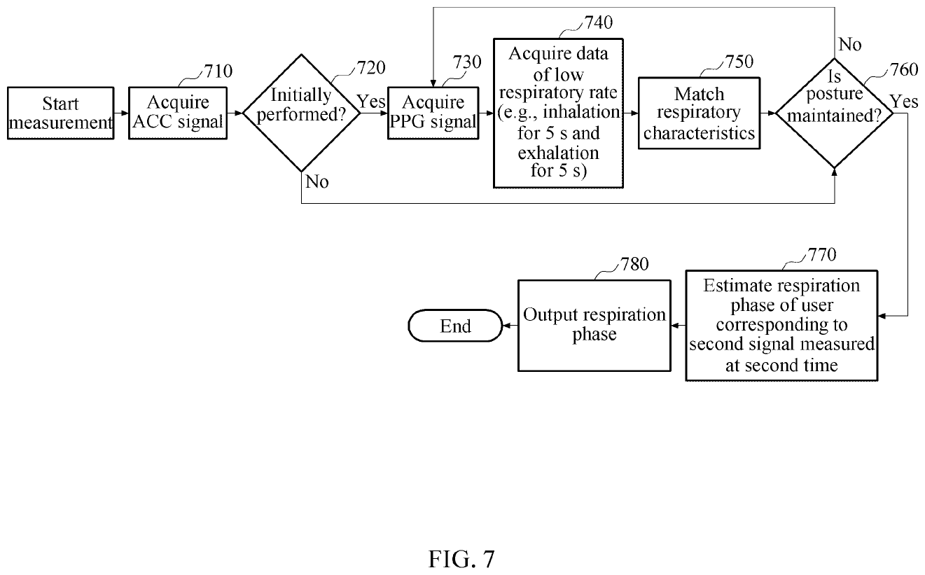

In an example, when the posture of the user at the second time remains the same as a posture of the user at the first time, the processor 430 may estimate the respiration phase of the user corresponding to the second time using the second signal measured at the second time based on the matched first and second respiratory characteristics. In another example, when the posture of the user at the second time does not remain the same as the posture of the user at the first time, the processor 430 may acquire a first signal corresponding to the second time, and match respiratory characteristics again based on a correlation between a change in the first signal corresponding to the second time and a change in the second signal corresponding to the second time. The processor 430 may match a first-second respiratory characteristic of the first signal corresponding to the second time and a second-second respiratory characteristic of the second signal corresponding to the second time, and estimate a respiration phase of the user corresponding to the second signal measured at the second time based on the matched first-second and second-second respiratory characteristics. A scheme by which the processor 430 estimates the respiration phase of the user based on whether the posture of the user is changed will be described in greater detail below with reference to FIG. 7.

The wearable electronic device 400 may further include a memory 450, an interface 470, and a display (e.g., the display 220 of FIG. 3).

The processor 430 may execute a program and control the wearable electronic device 400. A code of the program executed by the processor 430 may be stored in the memory 450.

The display 220 may display a respiration phase of a user estimated by the processor 430. The display 220 may be, for example, a touch display and/or a flexible display, but is not limited thereto.

The memory 450 may store signals or data received through the interface 470 and/or the respiration phase estimated by the processor 430.

The memory 450 may store a variety of information generated in a processing process of the processor 430 described above. In addition, the memory 450 may store a variety of data and programs. The memory 450 may include a volatile memory 450 or a non-volatile memory 450. The memory 450 may include a high-capacity storage medium such as a hard disk to store a variety of data.

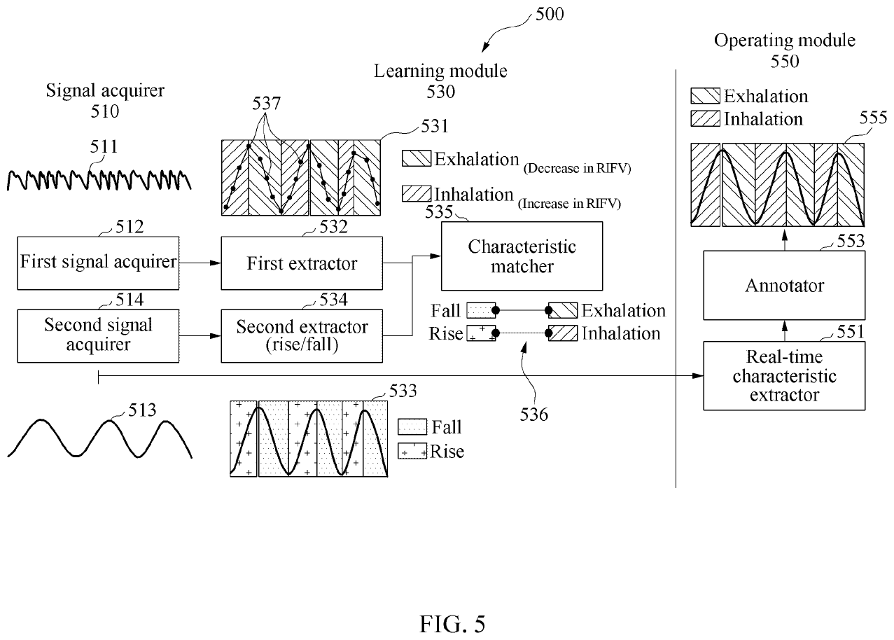

In addition, the processor 430 may perform at least one method that will be described with reference to FIGS. 5, 6, 7, 8 and 9 (which may be referred to as FIGS. 5 to 9) below, or a scheme corresponding to the at least one method. The wearable electronic device 400 may be a hardware-implemented wearable electronic device having a circuit that is physically structured to execute desired operations. For example, the desired operations may include codes or instructions included in a program. The hardware-implemented processor 430 may include, for example, a microprocessor, a CPU, a GPU, a processor core, a multi-core processor, a multiprocessor, an ASIC, a field-programmable gate array (FPGA), and/or a neural processing unit (NPU).

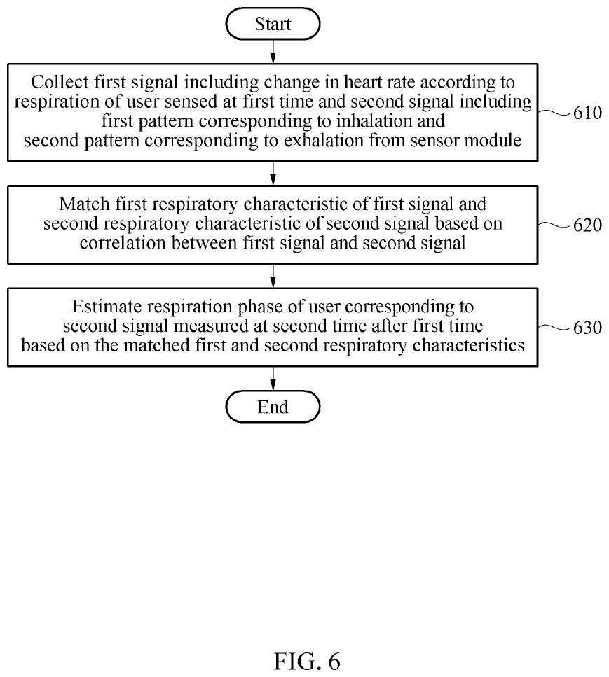

FIG. 5 is a diagram illustrating an example operation of a wearable electronic device according to an embodiment, and FIG. 6 is a flowchart illustrating an example method of operating a wearable electronic device according to an embodiment. In the following examples, operations may be performed sequentially, but not necessarily performed sequentially. For example, the order of the operations may be changed and at least two of the operations may be performed in parallel.

Referring to FIGS. 5 and 6, a wearable electronic device 500 (e.g., the electronic device 101 of FIG. 1, the electronic device 200 of FIGS. 2A and 2B, the electronic device 300 of FIG. 3, and/or the wearable electronic device 400 of FIG. 4) according to an embodiment may include a signal acquirer (e.g., including various circuitry) 510, a learning module (e.g., including various processing circuitry and/or executable program instructions) 530, and an operating module (e.g., including various processing circuitry and/or executable program instructions) 550, and may estimate a respiration phase of a user through operations 610, 620 and 630.

The signal acquirer 510 may include a first signal acquirer (e.g., including a sensor) 512 configured to acquire a first signal 511 (e.g., a PPG signal), and a second signal acquirer (e.g., including a sensor) 514 configured to acquire a second signal 513 (e.g., an ACC signal).

In operation 610, the signal acquirer 510 may collect a first signal including a change in a heart rate according to a respiration of a user sensed at a first time, and a second signal including a first pattern corresponding to inhalation and a second pattern corresponding to exhalation, from a sensor module (e.g., the sensor module 176 of FIG. 1, the sensor module 211 of FIGS. 2A and 2B, and/or the sensor module 410 of FIG. 4).

The learning module 530 may include various processing circuitry and/or executable program instructions and correspond to a configuration for connecting a direction of a change in the ACC signal 513 to inhalation or exhalation of the PPG signal 511 in an initial operation. The learning module 530 may include a first extractor 532, a second extractor 534, and a characteristic matcher 535, each of which may include various program instructions executed by various processing circuitry.

In operation 620, the learning module 530 may match a first respiratory characteristic of the first signal and a second respiratory characteristic of the second signal based on a correlation between the first signal and the second signal.

The first extractor 532 may sample the first signal, and may extract the first respiratory characteristic including an inhalation interval and an exhalation interval of the first signal, based on at least one of an interval in which an HRV by the sampled first signal increases, or an interval in which a stroke volume variation by the sampled first signal decreases. The first extractor 532 may extract a characteristic of the PPG signal 511 acquired by the first signal acquirer 512. The first extractor 532 may detect a change in the PPG signal 511 by the respiration from the PPG signal 511. For example, a physiological phenomenon in which a heart rate increases and in which a cardiac output decreases due to inspiration may occur. Due to the above physiological phenomenon, in a PPG signal, a respiratory-induced frequency variation (RIFV) may increase, and a respiratory-induced amplitude variation (RIAV) or a respiratory-induced intensity variation (RIIV)) may decrease. The first extractor 532 may identify and extract the first respiratory characteristic (e.g., inhalation and exhalation), based on the above-described direction of the change (e.g., an increase in the HRV or a decrease in the stroke volume variation).

The first extractor 532 may extract sampling points such as circular points 537 shown in a graph 531 showing a RIFV, may analyze a direction of a change by the extracted sampling points, and may distinguish between an interval in which the RIFV increases and an interval in which the RIFV decreases. Portions indicated by different patterns in the graph 531 may correspond to a result obtained by distinguishing between the interval in which the RIFV increases and the interval in which the RIFV decreases.

The second extractor 534 may extract a characteristic of the ACC signal 513. The second extractor 534 may extract a second respiratory characteristic (e.g., rise and fall) by identifying a phase of the ACC signal 513 acquired by the second signal acquirer 514 using a direction (e.g., a slope) of a change in the ACC signal 513. The phase of the ACC signal 513 may be used to match a rising interval to the inhalation or the exhalation in the characteristic matcher 535.

The second extractor 534 may determine a pattern corresponding to a rising interval in the second signal and a pattern corresponding to a falling interval in the second signal among the first pattern and the second pattern based on a slope of each of the first pattern and the second pattern, to determine the second respiratory characteristic (e.g., the rising interval and the falling interval).

An example in which the second extractor 534 extracts the second respiratory characteristic will be described below.

The second extractor 534 may generate a signal (e.g., aX+By+cZ) by one of X, Y, and Z components of the ACC signal 513 or a combination of two or more thereof. The second extractor 534 may detect and identify a direction of a change in a generated signal. In addition, the second extractor 534 may reduce baseline noise of an accelerometer with a frequency greater than a respiration frequency using a low-pass filter that blocks a frequency (e.g., 5 hertz (Hz)) greater than the respiration frequency.

The second extractor 534 may sample signals obtained by filtering out the baseline noise within an arbitrary time window (e.g., 100 milliseconds (ms)), and may distinguish between a rising interval and a falling interval of the ACC signal 513 based on a difference between the sampled signals (e.g., x(t−Δt₁₀₀ₘₛ), x(t), and x(t+Δt₁₀₀ₘₛ)). If the difference “x(t)−x(t−Δt₁₀₀ₘₛ)” between the sampled signals is greater than “0”, the second extractor 534 may determine a corresponding interval as a rising interval. If the difference “x(t)−x(t−Δt₁₀₀ₘₛ)” is less than or equal to “0”, the second extractor 534 may determine a corresponding interval as a falling interval.

For example, a measured value of an interval in which a respiration of the user is paused between inhalation and exhalation during the respiration may be inaccurate. The second extractor 534 may set a threshold so that a signal of a low slope interval (threshold interval) in which the user temporarily stops breathing may not be detected. The second extractor 534 may extract and identify respiratory characteristics of an ACC signal only when a slope of the ACC signal is greater than or equal to the threshold.

The characteristic matcher 535 may match the first respiratory characteristic and the second respiratory characteristic based on a comparison result between the first respiratory characteristic and the second respiratory characteristic. The characteristic matcher 535 may match portions corresponding to each other by comparing a direction of a change in the PPG signal to a direction of a change in the ACC signal. The characteristic matcher 535 may determine which of a rising interval and a falling interval of a graph 533 corresponding to the ACC signal 513 a portion determined as an inhalation interval in the graph 531 corresponding to the PPG signal 511 overlaps more. In FIG. 5, the inhalation interval of the graph 531 may overlap the rising interval of the graph 533, and the exhalation interval of the graph 531 may overlap the falling interval of the graph 533. Accordingly, the characteristic matcher 535 may perform a learning operation to match the rising interval of the ACC signal 513 to the inhalation interval of the PPG signal 511 and to match the falling interval of the ACC signal 513 to the exhalation interval of the PPG signal 511.

The learning module 530 according to an embodiment may guide the user to breathe at a relatively low respiratory rate for a more accurate connection between respiratory characteristics. This is because rising and falling phases of the ACC signal are most accurately matched to inhalation and exhalation detected by the PPG signal in response to a low respiratory rate, due to slowly sampling of the PPG signal.

For example, when inhaling for 5 seconds (s) and exhaling for 5 s is set as one cycle, the learning module 530 may guide the user to breathe for two cycles. The learning module 530 may match respiratory characteristics of signals (e.g., the first signal and the second signal) acquired according to the guiding.

If the matching of the respiratory characteristics is completed, the operating module 550 may distinguish a portion corresponding to the inhalation from a portion corresponding to the exhalation in the ACC signal 513 measured at the second time, based on information matched by the characteristic matcher 535.

In operation 630, the operating module 550 may estimate the respiration phase of the user corresponding to the second signal measured at the second time after the first time, based on the first respiratory characteristic and the second respiratory characteristic matched in operation 620. For example, the operating module 550 may extract a respiratory characteristic of the second signal measured at the second time after the first time, using a real-time characteristic extractor 551. The operating module 550 may add an annotation of inhalation or exhalation to respiratory characteristics extracted by the real-time characteristic extractor 551 based on the first respiratory characteristic and the second respiratory characteristic matched in operation 620, and estimate a respiration phase of the user corresponding to the second time.

The operating module 550 may output a respiration phase 555 of the user including inhalation and exhalation from the second signal (e.g., an ACC signal) measured at the second time (e.g., real time), based on a result obtained by matching respiratory characteristics of signals acquired at the first time in the learning module 530.

The operating module 550 may include, for example, the real-time characteristic extractor 551, and an annotator 553.

The real-time characteristic extractor 551 may extract respiratory characteristics from a second signal (e.g., an ACC signal) measured at a second time (e.g., real time).

The annotator 553 may add an annotation of inhalation or exhalation to the respiratory characteristics extracted by the real-time characteristic extractor 551 based on the first respiratory characteristic and the second respiratory characteristic matched in operation 620.

The annotator 553 may add an annotation on whether a respiratory characteristic extracted by the real-time characteristic extractor 551 corresponds to inhalation or exhalation, so that the wearable electronic device 500 may output the respiration phase 555 in a form of a graph, for example.

FIG. 7 is a flowchart illustrating an example method of operating a wearable electronic device according to an embodiment. In the following examples, operations may be performed sequentially, but not necessarily performed sequentially. For example, the order of the operations may be changed and at least two of the operations may be performed in parallel.

Referring to FIG. 7, a wearable electronic device (e.g., the electronic device 101 of FIG. 1, the electronic device 200 of FIGS. 2A and 2B, the electronic device 300 of FIG. 3, the wearable electronic device 400 of FIG. 4, and/or the wearable electronic device 500 of FIG. 5) according to an embodiment may output a respiration phase of a user through operations 710 to 780.

In operation 710, the wearable electronic device 400 may acquire an ACC signal.

In operation 720, the wearable electronic device 400 may determine whether operation 710 of acquiring the ACC signal is initially performed to measure a respiration phase. If operation 710 is initially performed, an artificial guide may be provided to obtain a low respiratory rate, or an induction to a state such as meditation or sleep may be possible.

If it is determined in operation 720 that operation 710 is initially performed, the wearable electronic device 400 may acquire a PPG signal in operation 730. If a condition for measuring a low respiratory rate is satisfied, the wearable electronic device 400 may acquire a PPG signal and store the acquired PPG signal in a memory. The wearable electronic device 400 may analyze results of biometric measurements (e.g., HR, stress, and SpO2) using a predetermined number of samples collected using a pulse wave sensor. For example, heartbeats may be measured with a relatively small number of samples, however, even a currently visible heartbeat may be estimated based on data for a few seconds previous to a current time.

In operation 740, the wearable electronic device 400 may acquire data of a low respiratory rate (e.g., a respiratory rate corresponding to inhalation performed for 5 s and exhalation performed for 5 s) from the PPG signal acquired in operation 730. The wearable electronic device 400 may store the PPG signal acquired in operation 730 in a memory on a premise that a condition for generation of the above guide or event is a low respiratory rate. If a time specified by the guide elapses, or if meditation or sleep ends, the wearable electronic device 400 may acquire data of the respiratory rate based on the data stored in the memory in operation 740.

In operation 750, the wearable electronic device 400 may match a first respiratory characteristic of the PPG signal acquired in operation 730 or 740 and a second respiratory characteristic of the ACC signal acquired in operation 710.

If matching of respiratory characteristics is completed in operation 750, the wearable electronic device 400 may determine whether a posture of the user is maintained in operation 760. In operation 760, the wearable electronic device 400 may determine whether the posture of the user is changed, based on an ACC signal measured at a second time. The wearable electronic device 400 may estimate a respiration phase of the user corresponding to the second time, based on whether the posture of the user is changed.

In operation 760, the wearable electronic device 400 may continue to monitor whether the posture of the user is maintained, based on the ACC signal. The wearable electronic device 400 may determine whether the posture of the user is changed, based on a comparison result between a third signal generated by an arbitrary combination of detailed signals included in the ACC signal and a threshold. For example, when a value of one of X, Y, and Z components of the ACC signal changes to be greater than or equal to a preset value, the wearable electronic device 400 may recognize that the posture of the user is changed. In addition, the wearable electronic device 400 may use an L2 norm using the X, Y, and Z components of the ACC signal, or use a vector dot product of the X, Y, and Z components of the ACC signal at a previous time (e.g., the first time or a time previous to the first time), to determine whether the posture of the user is changed.

In an example, if it is determined in operation 760 that the posture of the user is maintained, the wearable electronic device 400 may estimate the respiration phase of the user corresponding to the second signal measured at the second time, based on pre-learned information in operation 770. The wearable electronic device 400 may estimate the respiration phase of the user corresponding to the second time using the second signal measured at the second time based on the respiratory characteristics matched in operation 750. In operation 780, the wearable electronic device 400 may output the respiration phase estimated in operation 770.

In another example, if it is determined in operation 760 that the posture of the user is not maintained, the wearable electronic device 400 may acquire a PPG signal corresponding to the second time in operation 730 and guide rematching of respiratory characteristics, for re-learning. Guiding may be performed to restart a connection, and in operation 750, a first-second respiratory characteristic of the PPG signal corresponding to the second time and a second-second respiratory characteristic of the ACC signal corresponding to the second time may be matched based on a correlation between a change of the PPG signal corresponding to the second time and a change of the ACC signal corresponding to the second time.

If it is determined in operation 720 that operation 710 is not initially performed, the wearable electronic device 400 may perform operation 730 (if the posture is not maintained) or operation 770 (if the posture is maintained), depending on whether the posture of the user is maintained determined in operation 760.

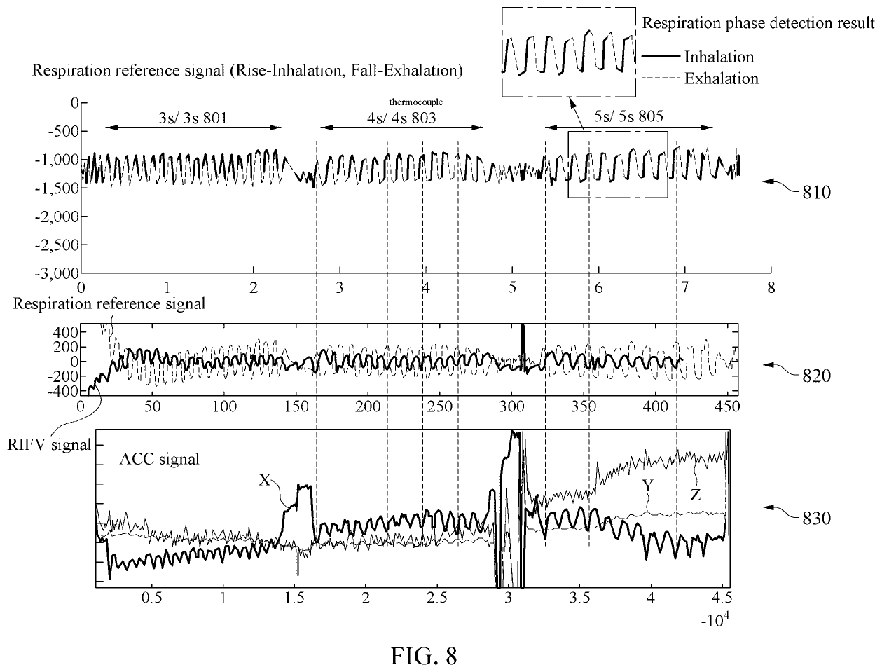

FIG. 8 illustrates graphs of signals measured at respiratory rates at different intervals in a wearable electronic device according to an embodiments. FIG. 8 illustrates a graph 810 showing respiration reference signals measured substantially simultaneously for three different respiratory rate intervals (e.g., a respiratory rate interval 801 with a pattern of 3 s/3 s, a respiratory rate interval 803 with a pattern of 4 s/4 s, and a respiratory rate interval 805 with a pattern of 5 s/5 s) in a wearable electronic device (e.g., the electronic device 101 of FIG. 1, the electronic device 200 of FIGS. 2A and 2B, the electronic device 300 of FIG. 3, the wearable electronic device 400 of FIG. 4, and/or the wearable electronic device 500 of FIG. 5), a graph 820 showing a RIFV signal in comparison to a reference signal, and a graph 830 showing an ACC signal including X, Y, and Z components.

The respiration reference signals shown in the graph 810 may be signals measured from a nose of a user with a thermocouple sensor. In the graph 810, a rising interval indicated by a solid line may represent “inhalation”, and a falling interval indicated by a dashed line may represent “exhalation”.

In the respiratory rate intervals 803 and 805 in the graph 820, it may be found that respiration reference signals indicated by dashed lines has phases opposite to that of the RIFV signal indicated by a solid line, and a correlation between the respiration reference signals is high. It may be found that an inhalation interval and an exhalation interval detected using the RIFV signal of the graph 820 are opposite to an inhalation interval and an exhalation interval of the respiration reference signal of the graph 810.

The above detection results may be more clearly understood from the respiratory rate interval 805 in the graph 830.

In the respiratory rate interval 805 in the graph 830, a rising interval of the ACC signal may correspond to an inhalation interval of the respiration reference signal, and a falling interval of the ACC signal may correspond to an exhalation interval of the respiration reference signal. However, in a relatively short respiration pattern such as the respiratory rate interval 801, it is difficult to clearly identify intervals of the ACC signal corresponding to the inhalation interval and the exhalation interval of the respiration reference signal. This is because a sampling interval of the RIFV signal is long, and the rising interval and the falling interval of the ACC signal and the inhalation interval and the exhalation interval of the respiration reference signal may be connected or matched by a relatively long respiration pattern such as the respiratory rate interval 805.

For example, in the case of a fast respiration such as the respiratory rate interval 801, inhalation and exhalation may be determined even with an increase or decrease of the ACC signal, and a respiration phase of a user may be estimated more accurately than a result obtained using only the PPG signal.

In an embodiment, an ACC signal for enabling a fast phase detection and a PPG signal for enabling an accurate phase detection may be matched and used, and thus it may be possible to more quickly and accurately detect the respiration phase for a fast respiratory rate as well as a slow respiratory rate.

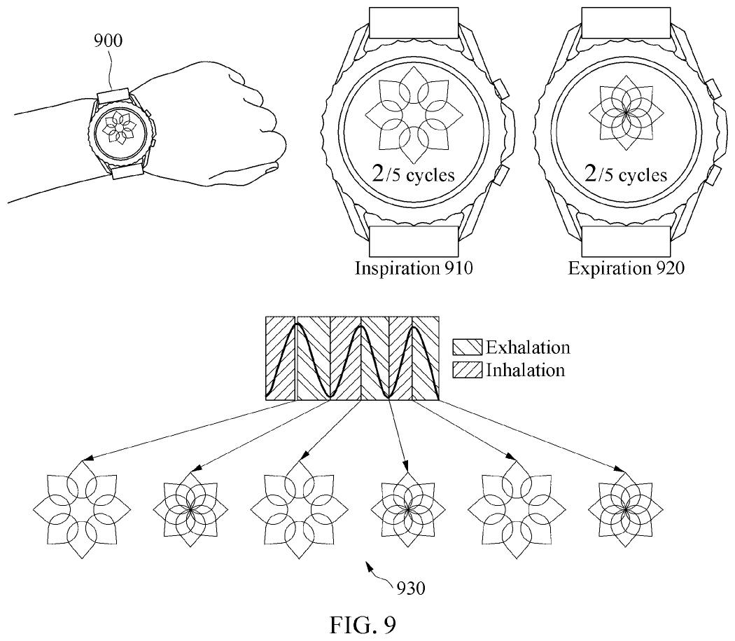

FIG. 9 is a diagram illustrating an example method of performing a breathing exercise using a wearable electronic device according to an embodiments. FIG. 9 illustrates screens 910 and 920 on which different graphic objects corresponding to a respiration phase of a user wearing a wearable electronic device 900 according to an embodiment are displayed, and an example 930 of a change in graphic objects according to a respiration of the user.

The wearable electronic device 900 may measure user's stress and inform the user of the stress, to manage mental health of modern people, or may also provide content associated with meditation or respiration as a mindfulness service for relieving stress. The breathing exercise may be recognized as an effective scheme to lower a heart rate and reduce stress by activating a parasympathetic nervous system, and meditation may also be generally based on various breathing methods.

The wearable electronic device 900 may help the user perform meditation and/or breathing by inducing respiration of the user through a graphic object (e.g., a lotus flower-shaped image) displayed on the screens 910 and 920.

The wearable electronic device 900 may display a lotus flower-shaped image on a screen while a size of the lotus flower-shaped image is repeatedly increasing or decreasing at regular intervals (e.g., 5 s) as shown in the example 930, to induce the user to breathe according to the graphic objects displayed on the screens 910 and 920. In the wearable electronic device 900, an ACC sensor may operate all the time, and a PPG sensor may sense inspiration (inhalation) and expiration (exhalation) of a user breathing five times per minute according to a meditation guide, for example.

If the meditation guide is provided, the wearable electronic device 900 may observe timings of inspiration (inhalation) and expiration (exhalation) of a user, and may adjust a display timing of an object displayed on a screen based on the observed timings. The wearable electronic device 900 may synchronize inspiration and expiration of a user sensed by a sensor (e.g., the first sensor 411 and the second sensor 412 of FIG. 4) with a graphic object displayed on a screen, and accordingly a deformation speed of the graphic object may be adjusted so that the user may slowly breathe in response to fast breathing.

For example, when a one-minute meditation guide ends, the wearable electronic device 900 may acquire respiratory rate data using a PPG signal and an ACC signal stored for one minute and may match respiratory characteristics.

The wearable electronic device 900 may measure stress, and provide a breathing exercise function as one of schemes to reduce the stress. By the breathing exercise function, inhalation and exhalation may be repeatedly performed a predetermined number of times at a predetermined time interval. The wearable electronic device 900 may provide, for example, a guide message such as “exhale” and/or “inhale”, and may give scores for breathing from 0 to 100 based on whether a user accurately performs inhaling and exhaling according to the guide message.

If the breathing exercise is performed in an open-loop manner, the wearable electronic device 900 may fail to provide feedback on whether a user properly performs the breathing exercise, but may synchronize inspiration and expiration of the user with a graphic object displayed on a screen. Thus, scoring of a respiration may be possible, a delay of estimating a respiration phase for a short respiration cycle may be reduced, and an accuracy may be enhanced.

According to an example embodiment, a wearable electronic device 101, 200, 300, 400, 500, 900 may include: a sensor module 176, 211, 410, including a first sensor 411 configured to sense a first signal 511 including a pulse wave based on a respiration corresponding to a first time, and a second sensor 412 configured to sense a second signal 513 including a first pattern corresponding to inhalation and a second pattern corresponding to exhalation, wherein the respiration may include the inhalation and the exhalation, and a processor 120, 430 configured to: match a first respiratory characteristic of the first signal 511 and a second respiratory characteristic of the second signal 513 based on a correlation between the first signal 511 and the second signal 513, and estimate a respiration phase corresponding to the second signal 513 measured at a second time after the first time based on the matched first and second respiratory characteristics.

According to an example embodiment, the processor 120, 430 may be configured to: extract the first respiratory characteristic from the first signal 511, extract the second respiratory characteristic from the second signal 513, and match the first respiratory characteristic and the second respiratory characteristic based on a comparison result between the first respiratory characteristic and the second respiratory characteristic.

According to an example embodiment, the processor 120, 430 may be configured to: sample the first signal 511, and extract the first respiratory characteristic including an inhalation interval and an exhalation interval of the first signal 511 based on at least one of an interval in which a heart rate variability (HRV) by the sampled first signal 511 increases or an interval in which a stroke volume variation by the sampled first signal 511 decreases.

According to an example embodiment, the processor 120, 430 may be configured to: determine a pattern corresponding to a rising interval in the second signal 513 and a pattern corresponding to a falling interval in the second signal 513 among the first pattern and the second pattern based on a slope of each of the first pattern and the second pattern.

According to an example embodiment, the processor 120, 430 may be configured to: match the first respiratory characteristic and the second respiratory characteristic based on which of the first pattern and the second pattern of the second signal 513 a portion of the first signal 511 determined as an inhalation interval overlaps more.

According to an example embodiment, the processor 120, 430 may be configured to: convert a rising interval of the second signal 513 measured at the second time to an inhalation interval and convert a falling interval of the second signal 513 measured at the second time to an exhalation interval based on the matched first and second respiratory characteristics, and estimate the respiration phase of the user based on the inhalation interval and the exhalation interval.

According to an example embodiment, the processor 120, 430 may be configured to: determine whether a posture is changed based on the second signal 513 measured at the second time, and estimate a respiration phase of the user corresponding to the second time based on whether the posture is changed.

According to an example embodiment, the processor 120, 430 may be configured to: determine whether the posture is changed, based on a comparison result between a third signal generated by an arbitrary combination of detailed signals included in the second signal 513 and a threshold.

According to an example embodiment, based on a posture at the second time remaining the same as a posture at the first time, the processor 120, 430 may be configured to: estimate the respiration phase corresponding to the second time, using the second signal 513 measured at the second time, based on the matched first and second respiratory characteristics.

According to an example embodiment, based on the posture at the second time not remaining the same as a posture at the first time, the processor 120, 430 may be configured to: acquire a first signal 511 corresponding to the second time, match a first-second respiratory characteristic of the first signal 511 corresponding to the second time and a second-second respiratory characteristic of a second signal 513 corresponding to the second time based on a correlation between a change in the first signal 511 corresponding to the second time and a change in the second signal 513 corresponding to the second time, and estimate the respiration phase of the user corresponding to the second signal 513 measured at the second time, based on the matched first-second and second-second respiratory characteristics.

According to an example embodiment, the second sensor 412 may include, for example, at least one of an acceleration sensor configured to sense a change in an acceleration based on a respiration and a movement, a gyro sensor configured to sense a change in a rotating angular speed based on the respiration and the movement, an acoustic sensor configured to sense sound corresponding to the inhalation and the exhalation based on the respiration, or an RF sensor configured to sense a change in a shape of a chest changed by the respiration, by an RF signal.

According to an example embodiment, a method of operating a wearable electronic device 101, 200, 300, 400, 500, 900 may include: operation 610 of collecting, from a sensor module 176, 211, 410, a first signal 511 including a change in a heart rate based on a respiration sensed at a first time and including inhalation and exhalation, and a second signal 513 including a first pattern corresponding to inhalation and a second pattern corresponding to exhalation, operation 620 of matching a first respiratory characteristic of the first signal 511 and a second respiratory characteristic of the second signal 513 based on a correlation between the first signal 511 and the second signal 513, and operation 630 of estimating a respiration phase corresponding to the second signal 513 measured at a second time after the first time based on the matched first and second respiratory characteristics.

According to an example embodiment, the matching of the first respiratory characteristic and the second respiratory characteristic may include: extracting the first respiratory characteristic from the first signal 511, extracting the second respiratory characteristic from the second signal 513, and matching the first respiratory characteristic and the second respiratory characteristic based on a comparison result between the first respiratory characteristic and the second respiratory characteristic.

According to an example embodiment, the extracting of the first respiratory characteristic may include: sampling the first signal 511, and extracting the first respiratory characteristic including an inhalation interval and an exhalation interval of the first signal 511 based on at least one of an interval in which a heart rate variability (HRV) by the sampled first signal 511 increases or an interval in which a stroke volume variation by the sampled first signal 511 decreases.

According to an example embodiment, the extracting of the second respiratory characteristic may include: determining a pattern corresponding to a rising interval in the second signal 513 and a pattern corresponding to a falling interval in the second signal 513 among the first pattern and the second pattern based on a slope of each of the first pattern and the second pattern.

According to an example embodiment, the matching of the first respiratory characteristic and the second respiratory characteristic may include: matching the first respiratory characteristic and the second respiratory characteristic based on which of the first pattern and the second pattern of the second signal 513 a portion of the first signal 511 determined as an inhalation interval overlaps more.

According to an embodiment, the estimating of the respiration phase of the user may include: converting a rising interval of the second signal 513 measured at the second time to an inhalation interval and converting a falling interval of the second signal 513 measured at the second time to an exhalation interval based on the matched first and second respiratory characteristics, and estimating the respiration phase of the user based on the inhalation interval and the exhalation interval.

According to an example embodiment, the estimating of the respiration phase may include: determining whether a posture is changed based on the second signal 513 measured at the second time, and estimating a respiration phase corresponding to the second time based on whether the posture is changed.

According to an example embodiment, the determining of whether the posture is changed may include: determining whether the posture is changed, based on a comparison result between a third signal generated by an arbitrary combination of detailed signals included in the second signal 513 and a threshold.

What is claimed is:

1. A wearable electronic device comprising:
a sensor module comprising a first sensor configured to sense a first signal comprising a pulse wave based on a respiration corresponding to a first time, the respiration comprising an inhalation and an exhalation, and a second sensor configured to sense a second signal comprising a first pattern corresponding to the inhalation and a second pattern corresponding to the exhalation; and
a processor configured to: match a first respiratory characteristic of the first signal and a second respiratory characteristic of the second signal based on a correlation between the first signal and the second signal, and estimate a respiration phase corresponding to the second signal measured at a second time after the first time, based on the matched first and second respiratory characteristics.

2. The wearable electronic device of claim 1, wherein the processor is configured to:
extract the first respiratory characteristic from the first signal;
extract the second respiratory characteristic from the second signal; and
match the first respiratory characteristic and the second respiratory characteristic based on a comparison result between the first respiratory characteristic and the second respiratory characteristic.

3. The wearable electronic device of claim 1, wherein the processor is configured to: sample the first signal and extract the first respiratory characteristic comprising an inhalation interval and an exhalation interval of the first signal based on at least one of an interval in which a heart rate variability (HRV) by the sampled first signal increases or an interval in which a stroke volume variation by the sampled first signal decreases.

4. The wearable electronic device of claim 1, wherein the processor is configured to: determine a pattern corresponding to a rising interval in the second signal and a pattern corresponding to a falling interval in the second signal among the first pattern and the second pattern based on a slope of each of the first pattern and the second pattern.

5. The wearable electronic device of claim 1, wherein the processor is configured to: match the first respiratory characteristic and the second respiratory characteristic based on which of the first pattern and the second pattern of the second signal a portion of the first signal determined as an inhalation interval overlaps more.

6. The wearable electronic device of claim 1, wherein the processor is configured to: convert a rising interval of the second signal measured at the second time to an inhalation interval and convert a falling interval of the second signal measured at the second time to an exhalation interval based on the matched first and second respiratory characteristics, and estimate the respiration phase based on the inhalation interval and the exhalation interval.

7. The wearable electronic device of claim 1, wherein the processor is configured to: determine whether a posture is changed based on the second signal measured at the second time, and estimate a respiration phase corresponding to the second time based on whether the posture is changed.

8. The wearable electronic device of claim 7, wherein the processor is configured to: determine whether the posture is changed, based on a comparison result between a third signal generated by an arbitrary combination of detailed signals included in the second signal and a threshold.

9. The wearable electronic device of claim 7, wherein the processor is configured to: estimate the respiration phase corresponding to the second time, using the second signal measured at the second time, based on the matched first and second respiratory characteristics, based on a posture at the second time remaining the same as a posture at the first time.

10. The wearable electronic device of claim 7, wherein the processor is configured to: acquire a first signal corresponding to the second time, match a first-second respiratory characteristic of the first signal corresponding to the second time and a second-second respiratory characteristic of a second signal corresponding to the second time based on a correlation between a change in the first signal corresponding to the second time and a change in the second signal corresponding to the second time, and estimate the respiration phase corresponding to the second signal measured at the second time, based on the matched first-second and second-second respiratory characteristics, based on the posture at the second time not remaining the same as a posture at the first time.

11. The wearable electronic device of claim 1, wherein the second sensor comprises at least one of:
an acceleration sensor configured to sense a change in an acceleration based on a respiration and a movement;
a gyro sensor configured to sense a change in a rotating angular speed based on respiration and movement;
an acoustic sensor configured to sense sound corresponding to inhalation and exhalation based on respiration; and
a radio frequency (RF) sensor configured to sense a change in a shape of a chest changed by respiration, by an RF signal.

12. A method of operating a wearable electronic device, the method comprising:
collecting, from a sensor module, a first signal comprising a change in a heart rate based on a respiration sensed at a first time, and a second signal comprising a first pattern corresponding to an inhalation and a second pattern corresponding to an exhalation, the respiration comprising the inhalation and the exhalation;
matching a first respiratory characteristic of the first signal and a second respiratory characteristic of the second signal based on a correlation between the first signal and the second signal; and
estimating a respiration phase corresponding to the second signal measured at a second time after the first time, based on the matched first and second respiratory characteristics.

13. The method of claim 12, wherein the matching of the first respiratory characteristic and the second respiratory characteristic comprises:
extracting the first respiratory characteristic from the first signal;
extracting the second respiratory characteristic from the second signal; and
matching the first respiratory characteristic and the second respiratory characteristic based on a comparison result between the first respiratory characteristic and the second respiratory characteristic.

14. The method of claim 12, wherein the extracting of the first respiratory characteristic comprises:
sampling the first signal; and
extracting the first respiratory characteristic comprising an inhalation interval and an exhalation interval of the first signal based on at least one of an interval in which a heart rate variability (HRV) by the sampled first signal increases or an interval in which a stroke volume variation by the sampled first signal decreases.

15. The method of claim 12, wherein the extracting of the second respiratory characteristic comprises: determining a pattern corresponding to a rising interval in the second signal and a pattern corresponding to a falling interval in the second signal among the first pattern and the second pattern based on a slope of each of the first pattern and the second pattern.

16. The method of claim 12, wherein the matching of the first respiratory characteristic and the second respiratory characteristic comprises: matching the first respiratory characteristic and the second respiratory characteristic based on which of the first pattern and the second pattern of the second signal a portion of the first signal determined as an inhalation interval overlaps more.

17. The method of claim 12, wherein the estimating of the respiration phase comprises:
converting a rising interval of the second signal measured at the second time to an inhalation interval and converting a falling interval of the second signal measured at the second time to an exhalation interval based on the matched first and second respiratory characteristics; and
estimating the respiration phase based on the inhalation interval and the exhalation interval.

18. The method of claim 12, wherein the estimating of the respiration phase comprises:
determining whether a posture is changed based on the second signal measured at the second time; and
estimating a respiration phase corresponding to the second time based on whether the posture is changed.

19. The method of claim 16, wherein the determining of whether the posture is changed comprises: determining whether the posture is changed, based on a comparison result between a third signal generated by an arbitrary combination of detailed signals included in the second signal and a threshold.

20. A non-transitory computer-readable storage medium storing instructions that, when executed by a processor, cause the processor to perform the operations of claim 12.
